(19) United States
(12) Patent Application Publication
(10) Pub. No.: US 2023/0355686 A1
(43) Pub. Date: Nov. 9, 2023

(54) METHOD FOR PREVENTING AND/OR TREATING HELICOBACTER PYLORI-RELATED DISEASE BY USING PARABACTEROIDES GOLDSTEINII AND ITS METABOLITES

(71) Applicant: Multistars Biotechnology Company Limited, Taoyuan City (TW)

(72) Inventors: Hsin-Chih Lai, Taoyuan City (TW); Chih-Ho Lai, Taoyuan City (TW); Chia-Chen Lu, Taoyuan City (TW); Tzu-Lung Lin, Taoyuan City (TW); Po-I Wu, Taoyuan City (TW)

(21) Appl. No.: 18/098,504

(22) Filed: Jan. 18, 2023

Related U.S. Application Data
(60) Provisional application No. 63/338,463, filed on May 5, 2022.

(30) Foreign Application Priority Data
Dec. 8, 2022 (TW) 111147277

(51) Int. Cl. A61K 35/74; A61K 45/06; A61P 31/04

(52) U.S. Cl. CPC A61K 35/74; A61K 45/06; A61P 31/04

(57) ABSTRACT

The present disclosure provides a method for preventing and/or treating Helicobacter pylori-related disease, including administering to a subject in need thereof a pharmaceutical composition including an effective amount of Parabacteroides goldsteinii and its metabolites, wherein the accession number for the Parabacteroides goldsteinii is DSM 32939. The Parabacteroides goldsteinii and its metabolites of the present disclosure reduces serum cholesterol level to achieve the effect of preventing and/or treating Helicobacter pylori-related disease.

### CROSS-REFERENCE TO RELATED APPLICATION

This application claims priorities of Provisional Application No. 63/338,463, filed on May 5, 2022, and Taiwan patent application No. 111147277, filed on Dec. 8, 2022, the content of which is incorporated herein in its entirety by reference.

### BACKGROUND OF THE INVENTION

### 1. Field of the Invention

The present invention relates to a method for preventing and/or treating Helicobacter pylori-related disease by using Parabacteroides goldsteinii and its metabolites.

### 2. The Prior Art

Persistent Helicobacter pylori (H. pylori) infection in the human stomach is closely related to the development of various gastrointestinal diseases, including chronic gastritis, peptic ulcers, and gastric cancer. Combination therapies using several antibiotics and a proton pump inhibitor (PPI) or bismuth are widely employed to eradicate H. pylori and relieve related gastrointestinal illnesses. However, a consequent increase in antibiotic resistance and changes in the gut microbiota often occur.

The two main virulence factors of H. pylori, vacuolating cytotoxin A (VacA) and cytotoxin-associated gene A (CagA), are involved in bacteria-induced pathogenesis. VacA is a pore-forming toxin that promotes acidic vacuole formation in host cells and inhibits mitochondrial function, resulting in apoptosis. CagA, which is encoded on the cag-pathogenicity island (cag-PAI), is delivered to host cells through the type IV secretion system (TFSS) and activates nuclear factor-kappa B (NF-κB) and IL-8 secretion, leading to inflammation and in some cases oncogenesis. Notably, these two virulence factors exploit cholesterol-rich microdomains in membranes, which are referred to as lipid rafts, for cell binding, intracellular delivery, and cell intoxication. Therefore, depleting cholesterol to disrupt lipid rafts is considered to be an ideal strategy to alleviate H. pylori-related diseases.

In view of the fact that the current drugs for the treatment of H. pylori-related diseases still have the disadvantages of side effects, cytotoxicity and ineffective effects, and the health products for preventing H. pylori-related diseases also have the disadvantages of ineffective effects, chemical synthesis and toxicity and side effects to individuals. In order to solve the above-mentioned problems, those skilled in the art urgently need to develop a novel and effective method for preventing and/or treating Helicobacter pylori-related disease for the benefit of a large group of people in need thereof.

### SUMMARY OF THE INVENTION

A primary objective of the present invention is to provide a method for preventing and/or treating Helicobacter pylori-related disease, comprising administering to a subject in need thereof a pharmaceutical composition comprising an effective amount of Parabacteroides goldsteinii and its metabolites, wherein the Parabacteroides goldsteinii is deposited in Deutsche Sammlung von Mikroorganismen and Zellkulturen (DSMZ) under an accession number DSM 32939.

According to an embodiment of the present invention, the Parabacteroides goldsteinii is a live bacterium.

According to an embodiment of the present invention, the effective amount of Parabacteroides goldsteinii is at least 5×10⁹ CFUs/kg per day for the subject.

According to an embodiment of the present invention, the Parabacteroides goldsteinii and its metabolites reduce serum cholesterol level.

According to an embodiment of the present invention, the Parabacteroides goldsteinii and its metabolites inhibit pathogenic effect of Helicobacter pylori vacuolating cytotoxin (VacA) and cytotoxin-associated gene A (CagA) in gastric epithelial cells.

According to an embodiment of the present invention, the pharmaceutical composition further comprises bacteria other than the Parabacteroides goldsteinii.

According to an embodiment of the present invention, the pharmaceutical composition is in a dosage form for oral administration.

According to an embodiment of the present invention, the pharmaceutical composition is in a dosage form for parenteral administration.

According to an embodiment of the present invention, the pharmaceutical composition further comprises a pharmaceutically acceptable excipient, carrier, adjuvant and/or food additive.

In summary, the Parabacteroides goldsteinii and its metabolites of the present invention have the effect on preventing and/or treating Helicobacter pylori-related disease by reducing serum cholesterol level.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the body weight and temperature of mice during experimental studies. Mice were divided into four groups for the treatments with vehicle-control (Ctl) (PBS, n=9), Parabacteroides goldsteinii (P. goldsteinii) MTS01 (PG, n=10), Helicobacter pylori (H. pylori) (HP, n=8), and P. goldsteinii MTS01+H. pylori (PG+HP) (n=10). The body weight and temperature of the mice were recorded every week for a total of twelve weeks.

FIG. 2 shows the murine model for experimental studies. Mice were divided into four groups for the treatments with vehicle-control (Ctl) (PBS, n=9), P. goldsteinii MTS01 (PG, n=10), H. pylori (HP, n=8), and P. goldsteinii MTS01+H. pylori (PG+HP) (n=10). Mice were intragastrically administered P. goldsteinii MTS01 (2×10⁸ CFU/100 μl) once daily for a total of nine weeks and continuously inoculated with P. goldsteinii MTS01 and H. pylori (1×10⁹ CFU/100 μl) once daily for additional two weeks. Mice were euthanized and the samples of blood, stomach, and stool were prepared for further analysis. IHC staining represents immunohistochemistry staining. CLO test represents Campylobacter-like organism test.

FIGS. 3A-3D show that P. goldsteinii MTS01 lowers serum triglyceride/cholesterol. Sera from mice (vehicle-control (Ctl), P. goldsteinii MTS01 (PG), H. pylori (HP), and P. goldsteinii MTS01+H. pylori (PG+HP)) were collected and the levels of (FIG. 3A) triglyceride, (FIG. 3B) total cholesterol, (FIG. 3C) low density lipoprotein (LDL)/very low density lipoprotein (VLDL), and (FIG. 3D) high density lipoprotein (HDL) were analyzed. *, P<0.05.

FIG. 4 shows that P. goldsteinii MTS01 mitigates H. pylori-induced gastric inflammation. Mouse (vehicle-control (Ctl), P. goldsteinii MTS01 (PG), H. pylori (HP), and P. goldsteinii MTS01+H. pylori (PG+HP)) gastric tissues were subjected to IHC staining with specific antibodies against cyclooxygenase-2 (COX-2), interleukin-1β (IL-1β), and tumor necrosis factor-α (TNF-α) (original magnification: 200×). The magnified images are displayed below each cropped area. Scale in each panel, 1000 μm, and in each magnified image, 100 μm. The intensity of COX-2, IL-1β, and TNF-α expression for IHC staining in gastric tissues are shown in the right panels.

FIGS. 5A-5D show that P. goldsteinii MTS01 ameliorates H. pylori-induced pathogenesis. (FIG. 5A) AGS cells were pretreated with P. goldsteinii MTS01 for 30 min and then infected with H. pylori at an MOI of 100 for 6 h. Cell vacuolation and elongation were observed by using a phase-contrast microscope. (FIG. 5B) The proportion of elongated cells were counted. (FIG. 5C) Cytotoxin-associated gene A (CagA) phosphorylation (p-CagA) were analyzed by western blot assay. Relative protein expression levels were normalized to β-actin and indicated under each lane. (FIG. 5D) The level of nuclear factor-kappa B (NF-κB) activation was assessed using luciferase assay. *, P<0.05. Ctl represents vehicle-control, PG represents P. goldsteinii MTS01, HP represents H. pylori.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

As used herein, the data provided represent experimental values that can vary within a range of ±20%, preferably within ±10%, and most preferably within ±5%.

As used herein, the term “effective dose” refers to the amount of Parabacteroides goldsteinii required for preventing and/or treating mammals or humans due to Helicobacter pylori-related disease. The effective dose may be different depending on the biological species or individual differences to be treated, but the effective dose can be determined experimentally by, for example, dose escalation.

According to the present invention, the operating procedures and parameter conditions related to bacterial culture fall within the scope of the professional literacy and routine techniques of those skilled in the art.

As used herein, the term “metabolite” means the substance secreted into the bacterial culture broth after being metabolized by the bacteria when the bacteria are cultured, including the culture broth of the bacteria.

As used herein, the term “bacterial component” means the derivative substances directly or indirectly related to the bacteria when the bacteria are cultured, including but not limited to the metabolites of the bacteria, the structure of the bacteria, active and inactive components related to the bacteria, etc.

As used herein, the term “treating” or “treatment” refers to alleviating, reducing, ameliorating, relieving or controlling one or more clinical signs of a disease or disorder, and lowering, stopping, or reversing the progression of severity regarding the condition or symptom being treated.

As used herein, the term “preventing” or “prevention” means preventing or delaying symptoms of disease onset when a drug is administered to an individual who does not have symptoms of disease onset but is at high risk of disease onset.

According to the present invention, the pharmaceutical composition can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

According to the present invention, the pharmaceutical composition may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar-containing solution, aqueous solution containing alcohol, and combinations thereof.

According to the present invention, probiotics and their metabolites play crucial roles in promoting the health of the host, including maintaining physiological homeostasis, improving intestinal integrity, and producing antimicrobial peptides against pathogens. Recently, several traditional probiotics have been applied to eradicate H. pylori infection and reduce the adverse effects of antibiotic-based therapy. However, the effectiveness of various probiotics in eradicating H. pylori is controversial. Given the conflicts regarding the clinical effectiveness of traditional probiotics, next generation probiotics (NGP), with fully defined genetic backgrounds and physiological characteristics that target specific diseases, have emerged.

According to the present invention, Parabacteroides goldsteinii MTS01, a newly discovered NGP, exerts multiple disease-alleviating functions, including obesity-reversing, insulin resistance-controlling, and chronic obstructive pulmonary disease (COPD) pathogenesis-ameliorating effects, by altering the gut microbiota. A recent study reported that loss of P. goldsteinii may in turn exacerbated colitis in model mice. Nevertheless, whether P. goldsteinii MTS01 can alter the microbiota ecosystem and mitigate H. pylori-induced pathogenesis in the stomach remains to be explored. In the present invention, we comprehensively investigated the mechanisms by which P. goldsteinii MTS01 inhibits H. pylori infection and ameliorates inflammation in the stomach using a murine model. Our results showed that administration of P. goldsteinii MTS01 in mice altered the composition of the gut microbiota and reduced serum cholesterol, which effectively attenuated the actions of H. pylori vacuolating cytotoxin (VacA) and cytotoxin-associated gene A (CagA). These findings suggest that P. goldsteinii MTS01 could potentially be developed as a functional probiotic against H. pylori-associated pathogenesis.

According to the present invention, differential abundance was analyzed using the Kruskal-Wallis to detect main effect differences and then applied to Wilcoxon rank-sum test for pairwise comparisons. Mann-Whitney U test was used to evaluate differences in microbiota abundance. In the biochemistry and cell-based studies, statistical significance was determined by Student's t-test between two groups. One-way ANOVA test with Tukey post-hoc test was used to assess the statistical significance of the experimental results for more than two studied groups. The statistical analysis was conducted using SPSS program (version 18.0 for Windows, SPSS Inc.) and the figures were performed by Prism program (version 9.0.0 for Windows, GraphPad Software). A P value less than 0.05 was considered statistically significant.

### Example 1

Bacterial Culture of Parabacteroides goldsteinii MTS01

The culture process of Parabacteroides goldsteinii (P. goldsteinii) MTS01 and Helicobacter pylori (H. pylori) strain 26695 (American Type Culture Collection (ATCC) 700392 is as follows. H. pylori strain 26695 was cultured on the blood agar plates (Brucella agar with 10% defibrinated sheep blood) and incubated at 37° C. in microaerophilic environment (5% O₂, 10% CO₂, and 85% N₂). P. goldsteinii MTS01 was isolated from the feces of a healthy adult who has taken P. goldsteinii MTS01 previously isolated from a healthy mouse for half a year. P. goldsteinii MTS01 was deposited in Deutsche Sammlung von Mikroorganismen and Zellkulturen (DSMZ) under an accession number DSM 32939, Oct. 29, 2018. It was cultured in thioglycollate medium (BD Biosciences) at 37° C. in an anaerobic chamber. Culture supernatant from P. goldsteinii MTS01 was filtrated using a 0.22 μm filter. Heat-inactivated P. goldsteinii MTS01 was prepared by boiling 1×10⁸ CFU/ml of bacterial suspension in PBS for 30 min. Live, heat-inactivated, and culture broth of P. goldsteinii MTS01 were collected for the following analysis.

In the embodiment of the present invention, the effective amount of Parabacteroides goldsteinii is at least 5×10⁹ CFUs/kg per day for the subject.

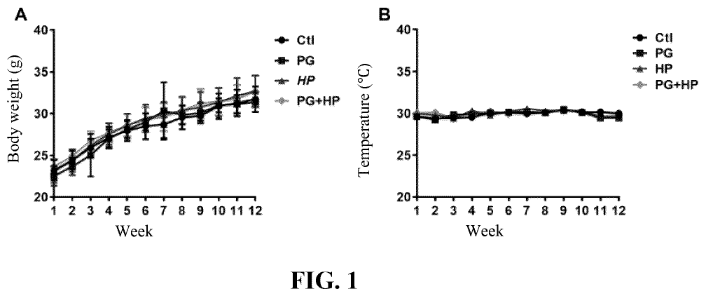

The operation process of the animal experiment is as follows. Six-week-old male BALB/c mice were purchased from the National Laboratory Animal Center (Taipei, Taiwan). The mouse experiments were performed in accordance with the Animal Care and Use Guidelines for Chang Gung University under a protocol approved by the Institutional Animal Care Use Committee (IACUC Approval No.: CGU107-141). The body weight and rectal temperature were recorded every morning during the study period (see FIG. 1).

FIG. 1 shows the body weight and temperature of mice during experimental studies. Mice were randomly divided into 4 groups for the treatments with vehicle control (Ctl) (PBS) (n=9), P. goldsteinii MTS01 (PG, n=10), H. pylori (HP, n=8), and P. goldsteinii MTS01+H. pylori (PG+HP, n=10), respectively. The body weight and temperature of the mice were recorded every week for a total of twelve weeks.

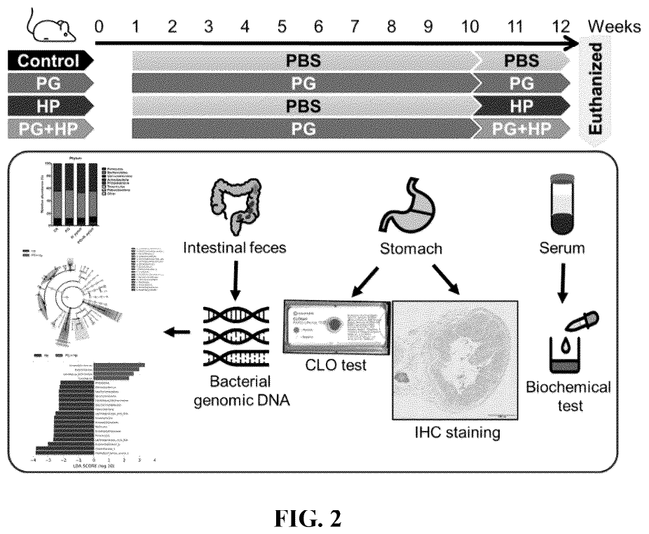

To assess the effects of P. goldsteinii MTS01 on H. pylori-induced gastric inflammation in vivo, mice were inoculated with live P. goldsteinii MTS01 via intragastric gavage prior to H. pylori infection. Mice were divided into the following four groups: vehicle control (Ctl) (PBS) (n=9), P. goldsteinii MTS01 (PG, n=10), H. pylori (HP, n=8), and P. goldsteinii MTS01+H. pylori (PG+HP, n=10), respectively (see FIG. 2).

FIG. 2 shows the murine model for experimental studies. Mice were intragastrically administered P. goldsteinii MTS01 (2×10⁸ CFU/100 μl) once daily for a total of nine weeks and continuously inoculated with P. goldsteinii MTS01 and H. pylori (1×10⁹ CFU/100 μl) once daily for additional two weeks. Mice were euthanized and the samples of blood, stomach, and stool were prepared for further analysis. IHC staining represents immunohistochemistry staining CLO test represents Campylobacter-like organism test.

### Example 2

Evaluation of Effect of Parabacteroides goldsteinii MTS01 on Reducing Serum Cholesterol Level and Mitigating Gastric Inflammation in H. pylori-Infected Mice

Because cholesterol is essential for the functions of H. pylori virulence factors, lowering cholesterol levels has been shown to mitigate host pathogenesis. To further investigate whether P. goldsteinii MTS01 alleviates H. pylori-induced pathogenesis mediated by reducing cholesterol levels, mouse serum triglyceride/cholesterol were analyzed, and gastric tissues were prepared for histopathological analysis.

The operation process of serum cholesterol/triglyceride analysis is as follows. Mouse blood was collected by cardiac puncture and centrifuged immediately (7000 rpm for 10 min) to prepare the serum. Serum triglyceride was analyzed using a Triglyceride Assay Kit (ab65336, Abcam). The levels of total cholesterol, high-density lipoprotein (HDL), low-density lipoprotein (LDL), and very-low-density lipoproteins (VLDL) in serum were assayed by a Cholesterol Assay enzyme linked immunosorbent assay (ELISA) Kit (ab65390, Abcam).

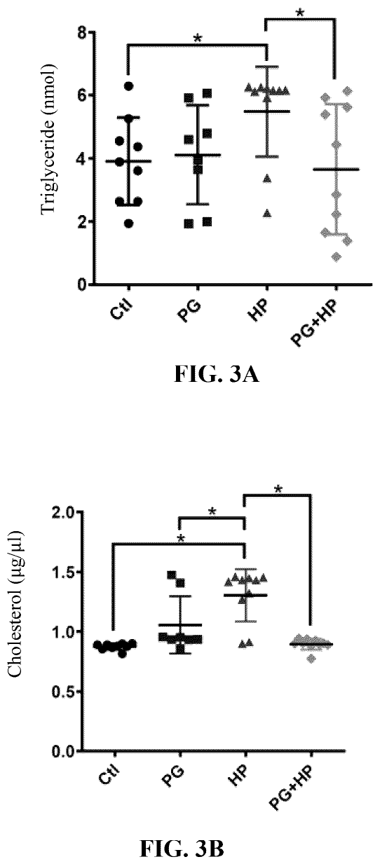

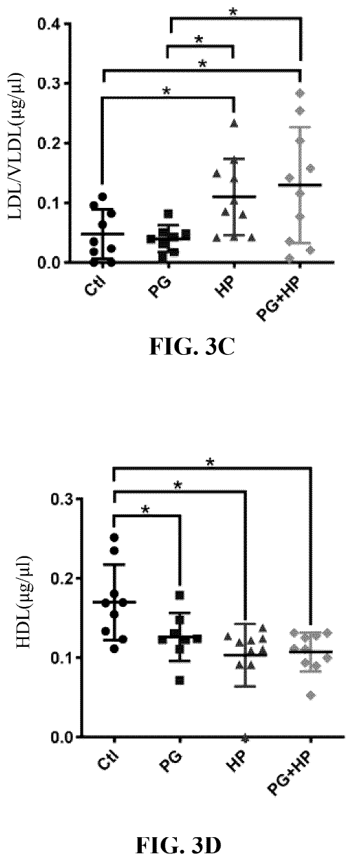

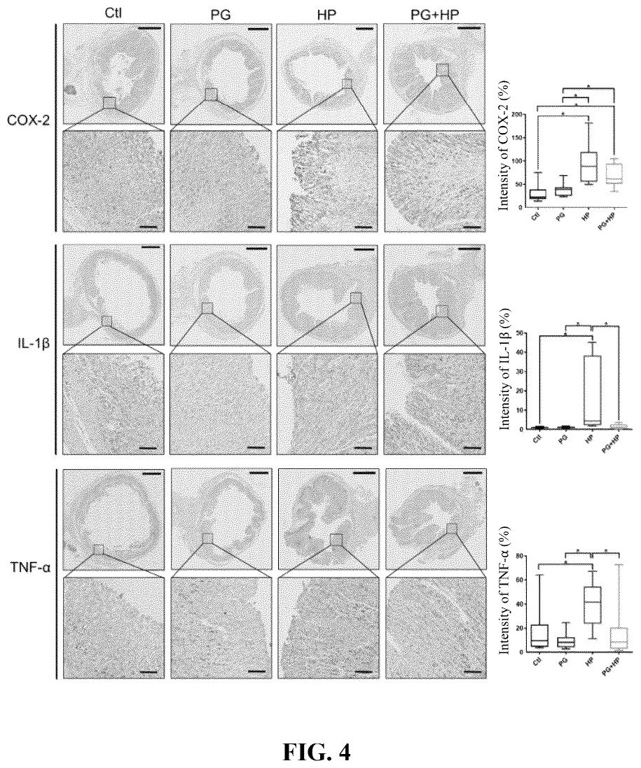

The operation process of histopathological analysis is as follows. Murine gastric tissues were prepared for immunohistochemistry (IHC) staining. The tissue sections were stained with monoclonal antibodies against cyclooxygenase-2 (COX-2), interleukin-1β (IL-1β), and tumor necrosis factor-α (TNF-α) (Abcam) at 4° C. for 18 h. After washing, the tissue sections were incubated with a horseradish peroxidase (HRP)-conjugated goat anti-rabbit secondary antibody (Epitomics) and developed with an ABC kit (Vector Laboratories). The stained tissues were analyzed by a histopathologist using a light microscope (Carl Zeiss). The results are shown in FIGS. 3A-3D and FIG. 4.

FIGS. 3A-3D show that P. goldsteinii MTS01 lowers serum triglyceride/cholesterol. Sera from mice (vehicle-control (Ctl), P. goldsteinii MTS01 (PG), H. pylori (HP), and P. goldsteinii MTS01+H. pylori (PG+HP)) were collected and the levels of (FIG. 3A) triglyceride, (FIG. 3B) total cholesterol, (FIG. 3C) low density lipoprotein (LDL)/very low density lipoprotein (VLDL), and (FIG. 3D) high density lipoprotein (HDL) were analyzed. *, P<0.05.

The result shows that serum levels of triglyceride and total cholesterol were significantly increased in mice infected with H. pylori compared to those in the control group (see FIGS. 3A and 3B). P. goldsteinii MTS01 treatment effectively inhibited these increases in serum triglyceride and cholesterol in H. pylori-infected mice but did not affect LDL/VLDL and HDL levels when compared to those in H. pylori-infected mice (see FIGS. 3C and 3D).

FIG. 4 shows that P. goldsteinii MTS01 mitigates H. pylori-induced gastric inflammation. Mouse (vehicle-control (Ctl), P. goldsteinii MTS01 (PG), H. pylori (HP), and P. goldsteinii MTS01+H. pylori (PG+HP)) gastric tissues were subjected to IHC staining with specific antibodies against COX-2, IL-1β, and TNF-α (original magnification: 200×). The magnified images are displayed below each cropped area. Scale in each panel, 1000 μm, and in each magnified image, 100 μm. The intensity of COX-2, IL-1β, and TNF-α expression for IHC staining in gastric tissues are shown in the right panels.

Mouse gastric tissues were further subjected to histopathological analysis. IHC analysis showed faint expression of proinflammatory cytokines (COX-2, IL-1β, and TNF-α) in the gastric tissues of control mice, indicating the absence of inflammation (see FIG. 4). In contrast, expression of these proinflammatory cytokines were much more pronounced in H. pylori-infected tissues but were remarkably reduced in those of H. pylori-infected mice fed P. goldsteinii MTS01. However, the Campylobacter-like organism (CLO) test indicates that H. pylori infection in stomach is unaffected by P. goldsteinii MTS01 treatment (data not shown). The operation process of the CLO test is as follows. CLO test (Kimberly Clark), a rapid urease test used to determine H. pylori infection. A gastric biopsy from mice was added into the CLO test microtube and incubated at room temperature for 3 h. The change in color of the gastric biopsies from yellow to red, which indicated the positive colonization of H. pylori.

Together, these results demonstrate that P. goldsteinii MTS01 treatment decreases serum cholesterol, which is associated with ameliorating the stomach inflammation caused by H. pylori.

### Example 3

Evaluation of Effect of Parabacteroides goldsteinii MTS01 on Alleviating H. pylori-Induced Pathogenesis

The two main virulence factors of H. pylori, VacA and CagA, exploit membrane cholesterol to exert their pathogenic effects on cells. In this example, we further investigated whether P. goldsteinii MTS01 lowers cholesterol and consequently attenuates VacA and CagA actions in cells. AGS cells were pretreated with P. goldsteinii MTS01 for 30 min and then infected with H. pylori for 6 h.

The culture process of AGS cells is as follows. AGS cells (ATCC CRL-1739, human gastric epithelial cell line) were cultured in F12 medium (Hyclone) containing 10% fetal bovine serum (Hyclone) and incubated at 37° C. in an incubator with 5% CO₂.

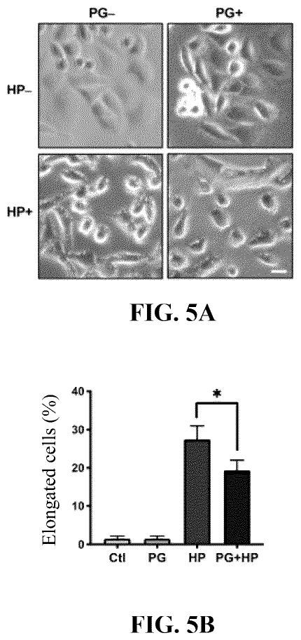

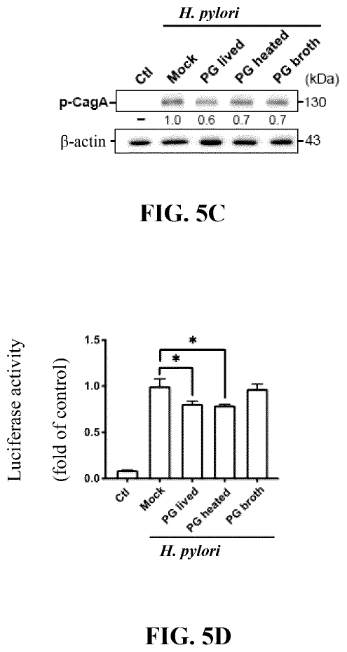

The operation process of determination of cell elongated phenotype is as follows. AGS cells were pretreated with P. goldsteinii MTS01 (MOI=200) followed by infection with H. pylori (MOI=100) for 6 h. H. pylori VacA-induced cell vacuolation and CagA-induced elongated cells (hummingbird phenotype) were observed. The percentage of elongated cells was determined. The result is shown in FIGS. 5A-5D.

FIGS. 5A-5D show that P. goldsteinii MTS01 ameliorates H. pylori-induced pathogenesis. (FIG. 5A) AGS cells were pretreated with P. goldsteinii MTS01 for 30 min and then infected with H. pylori at an MOI of 100 for 6 h. Cell vacuolation and elongation were observed by using a phase-contrast microscope. (FIG. 5B) The proportion of elongated cells were counted. (FIG. 5C) Cytotoxin-associated gene A (CagA) phosphorylation (p-CagA) were analyzed by western blot assay. Relative protein expression levels were normalized to β-actin and indicated under each lane. (FIG. 5D) The level of nuclear factor-kappa B (NF-κB) activation was assessed using luciferase assay. *, P<0.05. Ctl represents vehicle-control, PG represents P. goldsteinii MTS01, HP represents H. pylori.

As shown in FIG. 5A, H. pylori VacA-induced vacuole formation was effectively diminished in cells pretreated with P. goldsteinii MTS01. Similarly, the H. pylori CagA-induced scattering (hummingbird) phenotype was significantly decreased in P. goldsteinii MTS01-pretreated cells (see FIG. 5B).

We then investigated whether P. goldsteinii MTS01 inhibits H. pylori CagA phosphorylation in cells. The operation process of CagA phosphorylation assay is as follows. AGS cells were pretreated with P. goldsteinii MTS01 (MOI 200) for 30 min followed by infection with H. pylori (MOI=100) for additional 6 h. The cell extracts were prepared and subjected to 6% sodium dodecyl sulfate polyacrylamide gel electrophoresis (SDS-PAGE) then transferred onto polyvinylidene difluoride membranes (Millipore). The membranes were incubated with anti-phosphotyrosine (4G10) antibody (Millipore) overnight at 4° C., and then probed with a horseradish peroxidase-conjugated secondary antibody (Millipore). The expression level of phospho-CagA was analyzed using ECL western blotting detection reagents (GE Healthcare) and recorded by AzureSpot Analysis Software with Azure 400 (Azure Biosystems).

The operation process of NF-κB luciferase activity assay is as follows. AGS cells were transfected with NF-κB-luciferase reporter followed by treatment with P. goldsteinii MTS01 (MOI=200) and H. pylori (MOI=100) for 6 h. The cell lysates were prepared and subjected to luciferase assays using Dual-Luciferase Reporter Assay System (Promega). Luciferase activity was normalized by co-transfection of β-galactosidase expression vector (Promega).

The results showed that phosphorylation of CagA was increased in H. pylori-infected cells but was significantly decreased in cells pretreated with both live and heat-inactivated P. goldsteinii MTS01 (see FIG. 5C). This trend was also observed in an analysis of H. pylori-induced NF-κB activation (see FIG. 5D). Collectively, the results demonstrate that P. goldsteinii MTS01 has inhibitory activity against the pathogenic effects of H. pylori VacA and CagA in gastric epithelial cells, which may be attributed to its microbiota-altering and cholesterol-lowering effects.

In summary, the Parabacteroides goldsteinii and its metabolites of the present invention have the effect on preventing and/or treating Helicobacter pylori-related disease by reducing serum cholesterol level.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for preventing and/or treating Helicobacter pylori-related disease, comprising administering to a subject in need thereof a pharmaceutical composition comprising an effective amount of Parabacteroides goldsteinii and its metabolites, wherein the Parabacteroides goldsteinii is deposited in Deutsche Sammlung von Mikroorganismen and Zellkulturen (DSMZ) under an accession number DSM 32939.

2. The method according to claim 1, wherein the Parabacteroides goldsteinii is a live bacterium.

3. The method according to claim 1, wherein the effective amount of Parabacteroides goldsteinii is at least 5×10⁹ CFUs/kg per day for the subject.

4. The method according to claim 1, wherein the Parabacteroides goldsteinii and its metabolites reduce serum cholesterol level.

5. The method according to claim 1, wherein the Parabacteroides goldsteinii and its metabolites inhibit pathogenic effect of Helicobacter pylori vacuolating cytotoxin (VacA) and cytotoxin-associated gene A (CagA) in gastric epithelial cells.

6. The method according to claim 1, wherein the pharmaceutical composition further comprises bacteria other than the Parabacteroides goldsteinii.

7. The method according to claim 1, wherein the pharmaceutical composition is in a dosage form for oral administration.

8. The method according to claim 1, wherein the pharmaceutical composition is in a dosage form for parenteral administration.

9. The method according to claim 1, wherein the pharmaceutical composition further comprises a pharmaceutically acceptable excipient, carrier, adjuvant and/or food additive.
